(19) United States
(12) Patent Application Publication
(10) Pub. No.: US 2008/0039683 A1
(43) Pub. Date: Feb. 14, 2008

(54) Method of Using Thermochromic Devices

(76) Inventor: Joel L. Clupper, Woodland Hills, CA (US)

Correspondence Address: BERT P. KRAGES II;ATTORNEY AT LAW, 6665 SW HAMPTON ST, PORTLAND, OR, 97223, US

(21) Appl. No.: 11/875,721

(22) Filed: Oct. 19, 2007

Related U.S. Application Data
(62) Division of application No. 11/072,100, filed on Mar. 4, 2005.

(51) Int. Cl. A61F 5/00; G01K 11/00

(52) U.S. Cl. 600/38; 374/162

(57) ABSTRACT

A method of using a body cavity device of cylindrical, spherical, annular, or tubular form with thermochromic pigments that change color in response to temperature changes. The method can be used to induce stimulation within a body cavity and to discern the temperature within.

### CROSS-REFERENCED TO RELATED APPLICATIONS

This application is a divisional of U.S. application Ser. No. 11/072,100 filed on Mar. 4, 2005, which is incorporated by reference herein in its entirety.

### FIELD OF THE INVENTION

This invention relates to the use of body cavity devices to produce stimulation within body cavities such as the vagina or anus. The invention makes use of the temperature-indicating properties of thermochromic pigments.

### BACKGROUND OF THE INVENTION

The use of body cavity devices is well known in the prior art. A traditional application of such devices has been as sex aids used for the purpose of producing sexual stimulation and as prophylactics against pregnancy or disease transmission. When used as sexual aids or prophylactics, such devices are most commonly embodied in either: (1) a cylindrical form which is inserted into a cavity such as a vagina or anus and externally manipulated to produce stimulation, (2) in the form of one or more spherical or ovoid beads which are inserted into a cavity and from which stimulation is produced by rocking or otherwise moving the pelvic muscles, or (3) in the form of a sheath that covers an object to be inserted into the body cavity.

The use of thermochromic pigments in various applications is also well known. Such pigments typically comprise an electron-donating chromogenic substance, an electron-accepting substance, and a solvent which undergo a reversible color change in response to a change in the ambient temperature. The color change which takes place may be from one color to another, from colored to colorless, or from colorless to colored. Pigments may be combined so that several temperatures in a predetermined range can be indicated by different colors. Common commercial applications include the use in novelty jewelry, coffee mugs, fabrics, bank checks, and aquarium thermometers.

The temperature within the body cavities of humans, as well as other warm-blooded species, depends on factors such as activity level, ovulation, and sexual arousal. For example, the temperature of the vagina fluctuates during the day depending on the level of physical activity and food intake. It is generally accepted that the basal body temperature, i.e., the temperature upon awakening in the morning, can be used to indicate whether a female is ovulating. Before ovulation the temperature may vary from day to day by about 0.1 degree Celsius but just after ovulation the temperature rises by 0.2-0.6 degrees Celsius. The temperature changes experienced during sexual arousal vary among individuals depending on factors such as the level of vasocongestion in the tissues surrounding the genital region, the rate of metabolic energy expenditure, and the placement of the uterus relative to the cervix. E.g., Giulano, F., et. al., “Vaginal physiological changes in a model of sexual arousal in anesthetized rats,” Am J Physiol Regulatory Integrative Comp Physiol 2001, R140-R149; Henson, D. E., et. al., “Labial and Vaginal Blood Volume Responses to Visual and Tactile Stimuli,” Archives of Sexual Behavior, 1982, 11: 23-31; Abramson P. R., et. al., “Thermographic Measurement of Sexual Arousal: A Discriminant Validity Analysis,” 1981, Archives of Sexual Behavior, 10: 171-176.

### SUMMARY OF THE INVENTION

The invention consists of a method of inducing sexual stimulation using a body cavity device which has thermochromic pigments either embedded into or affixed onto so that they are visible when either an exterior or interior surface is viewed. The thermochromic pigments provide a means of indicating the temperature within a body cavity once the device is inserted and equilibrates in temperature with the cavity. The form of the body cavity device can be generally cylindrical, generally spherical, or a sheath depending on the configuration that is most suitable for the desired application.

The generally cylindrical embodiment of the body cavity device consists of a shaft of a diameter suitable for inserting into a body cavity with a first end and a second end. A conical head may be provided on the second end of the shaft to facilitate insertion and subsequent movement within the cavity. The shaft and head portions could feature ridges, differential diameters, and like protrusions as are well know in the prior art. The shaft and conical head can be fabricated of diverse materials with either rigid or semi-rigid properties. For example, rigid materials such as plastic or glass could have one or more thermochromic pigments bound within or applied in the form of a single or multi-layer coating system. Alternatively, the embodiment could be constructed of clear or translucent semi-rigid materials such as a silicone rubber in which the thermochromic pigment is incorporated. Alternatively, the embodiment in either a rigid or semi-rigid form could be constructed such that the thermochromic pigment is in a liquid or gel form is confined within a hollow space within the device.

The generally spherical embodiment of the body cavity device consists of one or more spherical or ovoid beads ranging about 10 to 40 millimeters in diameter. When this embodiment encompasses a plurality of beads, they can be connected by a cord or tube made of a suitable material such as nylon. The materials used to construct the beads and methods for incorporating or affixing the thermochromic pigment are the same as for the cylindrical embodiment.

The sheath embodiment of the body cavity device consists of a flexible ring or tubular body that can be slipped over an object intended to be inserted into a body cavity and thereby facilitate the taking of thermochromic measurements within such cavity. One such application is a thermochromic condom which would consist of a flexible tubular body having an open and a closed end formed of thin elastomeric material. Thermochromic pigments are bound within the material used to form the condom. However, the sheath embodiment could also be used to provide a cylindrical devices with a thermochromic measurement capability. For instance, It could also be fabricated as a ring or tube with two open ends and placed over a suitable object.

The thermochromic pigment can be incorporated into the body cavity device in a number of ways depending on the design and materials used to construct the invention. Thermochromic pigments are readily mixed with plastic or rubber formulations which are then forced into a mold to manufacture the invention. Other nonexclusive methods include: (1) incorporating the thermochromic pigment into a gel or liquid that fills a confined hollow space within the device and (2) coating the surface of the device with thermochromic coating that is further sealed with a top glaze of coating.

When the device is inserted into a body cavity, it is warmed by the heat within. The temperature within the cavity can be assessed visually by noting the color of the thermochromic pigment shortly after withdrawing the device. Alternatively, the device could feature a viewing window at the proximal end which allows a viewer to see into the device without the need for withdrawal from the cavity. A means for illuminating the internal portion of the device, such as a light emitting diode (LED), could be incorporated into the device to facilitate such viewing.

One application would be to use the temperature readings as a means of visual assessment of the degree of readiness for coitus. It is well established in the medical literature that sexually arousal in a woman correlates to the degree of blood engorgement and the production of transudate fluid in the vagina which are necessary precursors for comfortable coitus. Absent sufficient stimulation, the arousal process may not produce sufficient changes in the shape and lubricating abilities of the vagina to ensure a pleasant sexual experience. The cylindrical and spherical embodiments of the body cavity device provides the partner of the person undergoing foreplay stimulation with a means of providing stimulation while simultaneously monitoring temperature changes in the vagina that correlate to the level of vasocongestion present in the surrounding tissues. The temperature that best correlates to the preferred state of readiness can be determined through empirical experimentation and used as a guideline to ascertain when to commence coitus. Similarly, the invention could be used by a person engaged in autoerotic activity to provide feedback on changes within the measured cavity during various stages of perceived arousal.

When the body cavity device is embodied in the form of a condom, it is first warmed by the person wearing the device and is secondarily affected by the heat contributed by the other person upon penetration into the body cavity. The device used in this embodiment thus could provide a means of assessing body cavity temperature during coitus and similar activities, and overcomes a shortcoming in the prior art in which temperature measurements have been limited to unpartnered sexual stimulation. As discussed previously, the embodiments based on a sheath do not necessarily have to be condoms but could also take the form of a ring or sleeve with two open ends that slips over an object to be inserted into a body cavity. Such devices would allow users to modify existing objects to provide them with the capability of taking thermochromic measurements.

### BRIEF DESCRIPTION OF THE SEVERAL VIEWS OF THE DRAWINGS

A detailed description of the invention is hereafter described with specific reference being made to the drawings in which:

FIG. 1 is a perspective view of a cylindrical embodiment of the body cavity device;

FIG. 2 is a perspective view of an embodiment of the body cavity device showing a viewing port in the first end;

FIG. 3 is a side view of a spherical embodiment of the body cavity device showing two beads connected by a cord;

FIG. 4 is a side view showing an embodiment of the body cavity device as a condom.

### DETAILED DESCRIPTION OF THE INVENTION

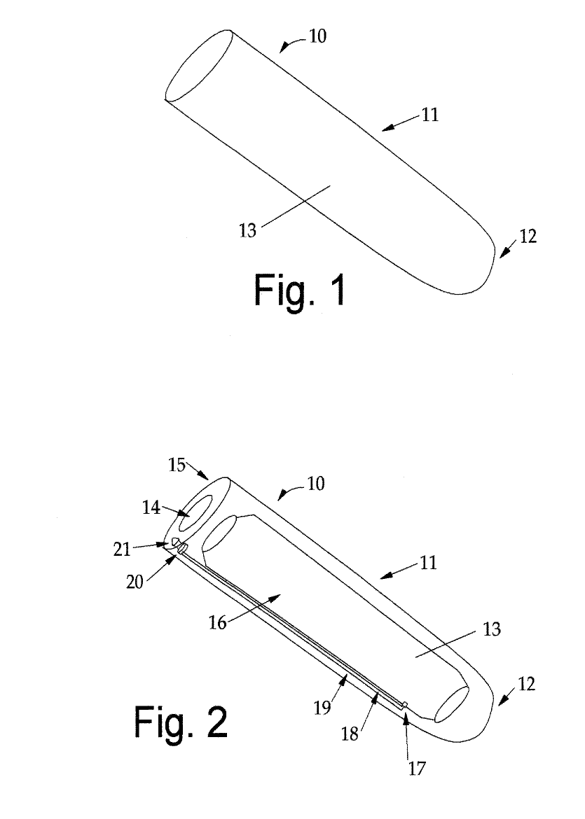

In the embodiment of the body cavity device shown in FIG. 1, the body cavity device is indicated generally by reference numeral 10. This embodiment consists of a generally cylindrical shaft 11 with a conical second end 12. The shaft and conical end may be made of any desired material but in the preferred embodiment would be a rigid material such as borosilicate glass, metal, or acrylic plastic. The shaft 11 and conical end 12 are covered with a thermochromic coating 13 which is affixed to the shaft in the form of a polymeric coating such as a polyacrylate that incorporates a thermochromic pigment. The thermochromic coating 13 changes color when a predetermined temperature is reached and may be provided with the ability to change to additional colors as the temperature further increases. Alternatively the shaft 11 and conical end 13 could be constructed of materials such as silicone, rubber, or semi-rigid thermoplastic in which the thermochromic pigment could be incorporated into the plastic resin or uncured rubber prior to injection molding or extrusion. The device 10 is used by insertion into a vagina or anus and manipulating the device so as to induce stimulation within the body cavity. The temperature within the cavity can be assessed by withdrawing the device 10 from the body cavity and visually observing the color of the thermochromic pigment 13.

The embodiment shown in FIG. 2. has a transparent port 14 in the base end 15 which enables viewing into a chamber 16 inside the shaft 11. The chamber is illuminated by a light emitting diode (LED) 17 which is connected by wires 18 and 19 to a battery 20 and switch 21 affixed to the base end 15. Pressing switch 21 activates the LED 17 which enables a viewer to look through the transparent port 15 and view the thermochromic pigment 13 lining the wall of the chamber 16. This allows a viewer to ascertain the body cavity temperature without having to remove the body cavity device 10 from the body cavity. Alternatively, the chamber 16 could be filled with a transparent or translucent liquid or semisolid gel which contains thermochromic pigment in which case the LED 17 would enable the viewer to ascertain the color of the thermochromic pigment by backlighting it.

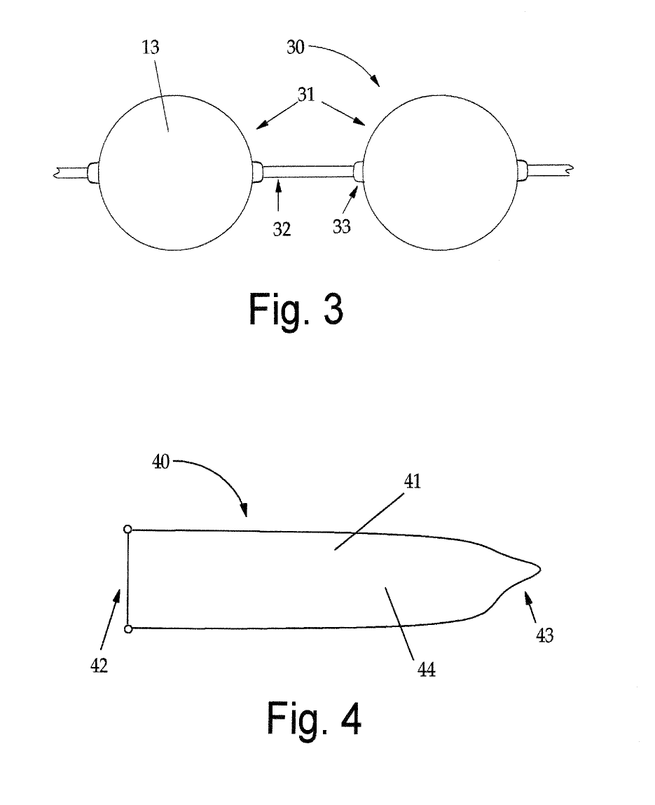

In the embodiment shown in FIG. 3, the body cavity device is indicated generally by reference numeral 30. This embodiment consists of one or more spherical beads 31 that may be affixed to a cord 32 running through the interior of the beads. The beads may be made of any desired material but in the preferred embodiment would be a hard material such as borosilicate glass, metal, or acrylic plastic. As is known in the art, the beads 31 are connected to the cord 32 by passing the cord through an opening formed through the interior of said beads. A knot 33, or other form of clasp, is secured to the cord 32 at each end of a bead 31 to prevent it from sliding along the length of the cord 32. The free end of the cord could have a handle portion in the form of a loop, ring, or the like. The beads 31 are covered with a thermochromic coating 13 which is affixed to the bead in the form of a polymeric coating such as a polyacrylate that incorporates a thermochromic pigment. The thermochromic coating 13 changes color when a predetermined temperature is reached and may be provided with the ability to change to additional colors as the temperature further increases. Alternatively the beads 31 could be constructed of materials such as silicone, rubber, or thermoplastics in which the thermochromic pigment is incorporated into the plastic resin or uncured rubber prior to injection molding or extrusion. The device 30 is used by insertion into a vagina or anus and manipulating the device 30 by rocking or otherwise moving the pelvic muscles so as to induce stimulation within the body cavity. The temperature within the cavity can be assessed by withdrawing the device 30 from the body cavity and visually observing the color of the thermochromic pigment 13.

In the embodiment of the body cavity device shown in FIG. 4, the body cavity device is indicated generally by reference numeral 40. This embodiment consists of a thin elastic sheath 41 of a generally cylindrical or tubular configuration and having an open end 42 and a rounded closed end 43. The sheath 41 consists of a conventional material such as latex, urethane, rubber or the like, that has thermochromic pigments 44 embedded therein. An alternative embodiment, not shown, would have two open ends and be suitable for using as a thermochromic sleeve for covering a non-thermochromic body cavity device or a similarly shaped object to give them the ability to take thermochromic measurements when inserted into a body cavity. The device 40 is used by placing it over a substantially-cylindrical object to form an assembly which is inserted into a vagina or anus and thereafter manipulated so as to induce stimulation within the body cavity. The temperature within the cavity can be assessed by withdrawing the assembly comprising the device 40 from the body cavity and visually observing the color of the thermochromic pigment 44.

The above examples and disclosures are intended to be illustrative and not exhaustive. These examples and description will suggest many variations and alternatives to one of ordinary skill in this art. All these alternatives and variations are intended to be included within the scope of the attached claims. Those familiar with the art may recognize other equivalents to the specific embodiments described herein, which equivalents are also intended to be encompassed by the claims attached hereto. With respect to the above description then, it is to be realized that the optimum dimensional relationships for the parts of the body cavity device, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present body cavity device. Because numerous modifications and changes will readily occur to those skilled in the art, it is not desired to limit the body cavity device to the exact construction and operation shown and described, and accordingly, all suitable modifications and equivalents may be resorted to, falling within the scope of the method of using a body cavity device.

I claim:

1. A method of inducing sexual stimulation and visual assessment, the method including:
a device comprising at least one thermochromic pigment that exhibits a predetermined detectable change in color when the thermochromic pigment is in contact with an object having a predetermined temperature;
the step of the inserting said device into a vagina or anus;
and further comprising the step of manipulating said device to produce sexual stimulation;
and further comprising the step of viewing the color of said thermochromic pigment.

2. The method of claim 1 in which said device further comprises an elongated, generally cylindrical shaft constructed of sufficiently rigid material so as to minimize bending when said shaft is inserted into said vagina or anus.

3. The method of claim 1 in which in said device further comprises:
an elongated, generally cylindrical shaft constructed of sufficiently rigid material so as to minimize bending when said shaft is inserted into said vagina or anus; and
in which said thermochromic pigment is affixed to the outer surface of the shaft.

4. The method of claim 1 in which in said device further comprises:
an elongated, generally cylindrical shaft constructed of sufficiently rigid material so as to minimize bending when said shaft is inserted into said vagina or anus; and
in which said thermochromic pigment is embodied into the material comprising the shaft.

5. The method of claim 1 in which in said device further comprises:
an elongated, generally cylindrical shaft constructed of sufficiently rigid material so as to minimize bending when said shaft is inserted into said vagina or anus;
said thermochromic pigment located within a hollow space within the shaft; and
said shaft has a transparent port for viewing said thermochromic pigment.

6. The method of claim 1 in which in said device further comprises:
an elongated, generally cylindrical shaft constructed of sufficiently rigid material so as to minimize bending when said shaft is inserted into said vagina or anus;
said thermochromic pigment is within a hollow space within the shaft;
said shaft has a transparent port for viewing said thermochromic pigment; and
a light source located within the shaft that is selectively operated by a switch.

7. The method of claim 1 in which in said device further comprises one or more generally spherical elements being constructed of sufficiently rigid material so as to minimize collapse when inserted into said vagina or anus.

8. The method of claim 1 in which in said device further comprises:
one or more generally spherical elements being constructed of sufficiently rigid material so as to minimize collapse when inserted into said vagina or anus; and
in which said thermochromic pigment is affixed to the outer surface of at least one said element.

9. The method of claim 1 in which in said device further comprises:
one or more generally spherical elements being constructed of sufficiently rigid material so as to minimize collapse when inserted into said vagina or anus; and
the thermochromic pigment is embodied into the material comprising an element

10. The method of claim 1 in which in said device further comprises:
one or more generally spherical elements being constructed of sufficiently rigid material so as to minimize collapse when inserted into said vagina or anus; and
said thermochromic pigment is confined within a hollow space within the wall of at least one of said elements and is viewable through a transparent portion of the wall of the element.

11. The method of claim 1 in which in said device further comprises:
a plurality of generally spherical elements being constructed of sufficiently rigid material so as to minimize collapse when inserted into said vagina or anus; and
said elements are connected by a flexible cord.

12. A method of inducing sexual stimulation and visual assessment, the method including:
the step of inserting an assembly into a vagina or anus;
in which said assembly comprises:
a round flexible hollow body with thermochromic pigment that exhibits a predetermined detectable change in color when the thermochromic pigment is in contact with an object having a predetermined temperature; and
a generally cylindrical shaft having sufficiently rigid material so as to minimize bending when said shaft is inserted into said vagina or anus; and
in which said body is placed over said generally cylindrical shaft;
further comprising the step of manipulating said assembly to produce sexual stimulation;
and further comprising the step of viewing the color of said thermochromic pigment.

13. The method of claim 12 in which said body comprises a membrane having a generally constant diameter from the open end to the other end to define a longitudinally-extending tubular shape.

14. The method of claim 12 in which said body:
comprises a membrane having a generally constant diameter from the open end to the other end to define a longitudinally-extending tubular shape; and
in which said thermochromic pigment is immobilized onto the membrane.

15. The method of claim 12 in which said body:
comprises a membrane having a generally constant diameter from the open end to the other end to define a longitudinally-extending tubular body; and
in which said thermochromic pigment is embodied within the membrane.

16. The method of claim 12 in which said body further comprises a membrane having an open end and a closed end, and further having a generally constant diameter from the open end to the closed end to define a longitudinally-extending tubular sheath.

17. The method of claim 12 in which said body further comprises:
a membrane having an open end and a closed end, and further having a generally constant diameter from the open end to the closed end to define a longitudinally-extending tubular sheath; and
in which said thermochromic pigment is immobilized onto the membrane

18. The method of claim 12 in which said body further comprises:
a membrane having an open end and a closed end, and further having a generally constant diameter from the open end to the closed end to define a longitudinally-extending tubular sheath; and
in which said thermochromic pigment is embodied in the membrane.
